(19) United States
(12) Patent Application Publication
(10) Pub. No.: US 2026/0041559 A1
(43) Pub. Date: Feb. 12, 2026

(54) UNICOMPARTMENTAL ARTHROPLASTY FEMORAL COMPONENT

(71) Applicant: Yi-Loong Colin Woon, Fort Lee, NJ (US)

(72) Inventor: Yi-Loong Colin Woon, Fort Lee, NJ (US)

(21) Appl. No.: 19/294,905

(22) Filed: Aug. 8, 2025

Related U.S. Application Data
(60) Provisional application No. 63/681,357, filed on Aug. 9, 2024.

(51) Int. Cl. A61F 2/38; A61F 2/30

(52) U.S. Cl. CPC A61F 2/3859; A61F 2002/30131; A61F 2002/30878; A61F 2002/3895

(57) ABSTRACT

A unicompartmental arthroplasty femoral component comprising a main body and a plurality of pegs that extend from the posterior surface. The main body has an anterior surface, a posterior surface, a proximal end joining the anterior surface and the posterior surface, and a distal end joining the anterior surface and the posterior surface. The anterior surface has a crescent shape and an anterior intermediate point disposed between the proximal end and the distal end. The proximal end and the distal end are disposed on a first line that lies on a first plane that is parallel to a second plane that includes a second line that is tangent to the anterior intermediate point. The posterior surface defining a plurality of pairs of facets.

### FIELD

The disclosure relates to the field of orthopedic implants. More particularly, the disclosure relates to unicompartmental femoral components for use in arthroplasty procedures.

### BACKGROUND

Traditional unicompartmental arthroplasty femoral components present limitations, including potential complications during surgery and postoperative challenges related to implant positioning and stability.

A need exists, therefore, for improved unicompartmental arthroplasty femoral components.

### BRIEF SUMMARY OF SELECTED EXAMPLES

Various example unicompartmental arthroplasty femoral components are described.

An example unicompartmental arthroplasty femoral component comprises a main body. The main body has an anterior surface, a posterior surface, a proximal end joining the anterior surface and the posterior surface, and a distal end joining the anterior surface and the posterior surface. The anterior surface has a crescent shape and an anterior intermediate point disposed between the proximal end and the distal end. The proximal end and distal end are disposed on a first line that lies on a first plane that is parallel to a second plane that includes a second line that is tangent to the anterior intermediate point. The posterior surface defines a plurality of pairs of facets. Each facet of a first pair of facts of the plurality of pairs of facets extends from the proximal end to a first posterior intermediate point that is between the proximal end and the distal end. Each facet of a second pair of facets of the plurality of pairs of facets extends from the distal end to a second posterior intermediate point that is between the proximal end and the distal end. Each facet of a third pair of facets of the plurality of pairs of facets extends from the first posterior intermediate point or the second posterior intermediate point to a point on a third plane that is parallel to the second plane. A plurality of pegs extend from the posterior surface.

Another example unicompartmental arthroplasty femoral component comprises a main body. The main body has an anterior surface, a posterior surface, a proximal end joining the anterior surface and the posterior surface, and a distal end joining the anterior surface and the posterior surface. The anterior surface has a crescent shape and an anterior intermediate point disposed between the proximal end and the distal end. The proximal end and the distal end are disposed on a first line that lies on a first plane that is parallel to a second plane that includes a second line that is tangent to the anterior intermediate point. The posterior surface has a posterior surface perimeter, the posterior surface defines a plurality of pairs of facets and a lip, each facet of a first pair of facets of the plurality of pairs of facets extends from the proximal end to a first posterior intermediate point that is between the proximal end and the distal end. Each facet of a second pair of facets of the plurality of pairs of facets extends from the distal end to a second posterior intermediate point that is between the proximal end and the distal end. Each facet of a third pair of facets of the plurality of pairs of facets extends from the first posterior intermediate point or the second posterior intermediate point to a point on a third plane that is parallel to the second plane. A plurality of pegs extend from the posterior surface.

Another example unicompartmental arthroplasty femoral component comprises a main body. The main body has an anterior surface, a posterior surface, a proximal end joining the anterior surface and the posterior surface, and a distal end joining the anterior surface and the posterior surface. The anterior surface has a crescent shape and an anterior intermediate point disposed between the proximal end and the distal end. The proximal end and the distal end are disposed on a first line that lies on a first plane that is parallel to a second plane that includes a second line that is tangent to the anterior intermediate point. The posterior surface defines a plurality of pairs of facets, each facet of a first pair of facets of the plurality of pairs of facets extends from the proximal end to a first posterior point that is between the proximal end and the distal end. Each facet of a second pair of facets of the plurality of pairs of facets extend from the distal end to a second posterior intermediate point that is between the proximal end and the distal end, each facet of a third pair of facets of the plurality of pairs of facets extends from the first posterior intermediate point or the second posterior intermediate point to a point on a third plane that is parallel to the second plane. A plurality of pegs extend from the posterior portion. At least one of the plurality of pegs is offset from the others.

Additional understanding of the inventive unicompartmental arthroplasty femoral components can be obtained by reviewing the detailed description of selected examples, below, and the referenced drawings.

### DESCRIPTION OF FIGURES

FIG. 1 is a perspective view of an example unicompartmental arthroplasty femoral component.

FIG. 2 is a perspective view of a posterior surface of the example unicompartmental arthroplasty femoral component illustrated in FIG. 1.

FIG. 3 is a flat plan view of a side of the example unicompartmental arthroplasty femoral component illustrated in FIG. 1.

FIG. 4 is a perspective view of an example unicompartmental arthroplasty femoral component.

FIG. 5 is a perspective view of a posterior surface of the example unicompartmental arthroplasty femoral component illustrated in FIG. 4.

FIG. 6 is a flat plan view of a side of the example unicompartmental arthroplasty femoral component illustrated in FIG. 4.

FIG. 7 is a perspective view of an example unicompartmental arthroplasty femoral component.

FIG. 8 is a perspective view of a posterior surface of the example unicompartmental arthroplasty femoral component illustrated in FIG. 7.

FIG. 9 is a flat plan view of a side of the example unicompartmental arthroplasty femoral component illustrated in FIG. 7.

FIG. 10 is a perspective view of an example unicompartmental arthroplasty femoral component.

FIG. 11 is a perspective view of a posterior surface of the example unicompartmental arthroplasty femoral component illustrated in FIG. 10.

FIG. 12 is a flat plan view of a side of the example unicompartmental arthroplasty femoral component illustrated in FIG. 10.

FIG. 13 is a perspective view of an example unicompartmental arthroplasty femoral component.

FIG. 14 is a perspective view of a posterior surface of the example unicompartmental arthroplasty femoral component illustrated in FIG. 13.

FIG. 15 is a flat plan view of a side of the example unicompartmental arthroplasty femoral component illustrated in FIG. 13.

FIG. 16 is a perspective view of an example unicompartmental arthroplasty femoral component.

FIG. 17 is a perspective view of a posterior surface of the example unicompartmental arthroplasty femoral component illustrated in FIG. 16.

FIG. 18 is a flat plan view of a side of the example unicompartmental arthroplasty femoral component illustrated in FIG. 16.

### DETAILED DESCRIPTION OF SELECTED EXAMPLES

The following detailed description and the appended drawings describe and illustrate various example unicompartmental arthroplasty femoral components. The description and illustration of these examples enable one skilled in the art to make and use examples of unicompartmental arthroplasty femoral components. They do not limit the scope of the claims in any manner.

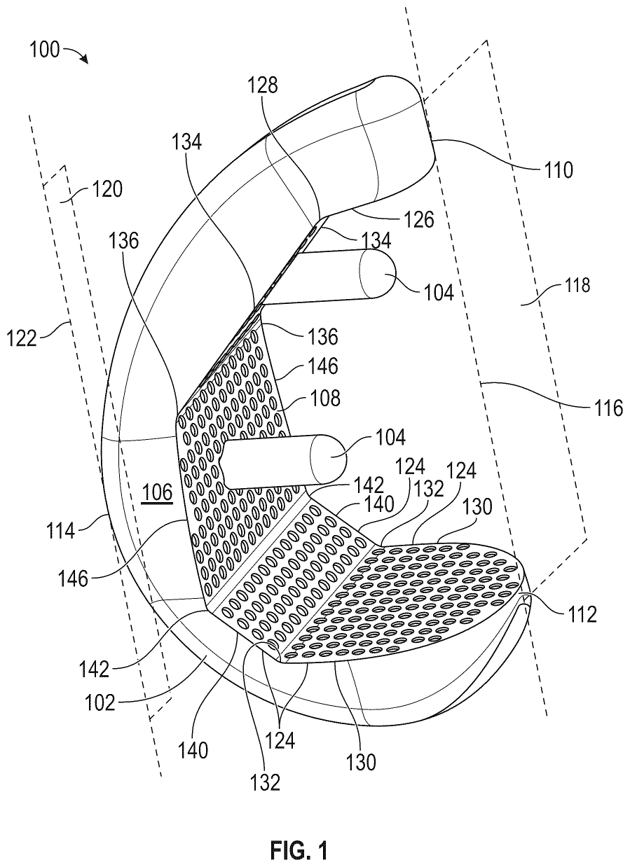

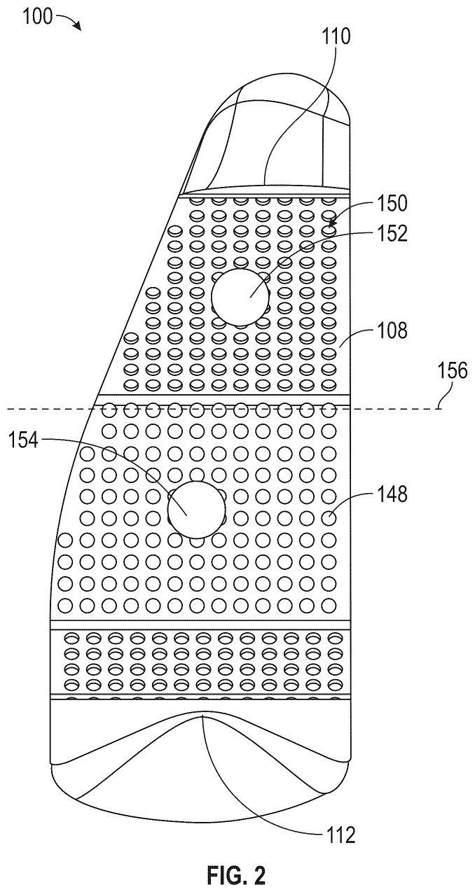

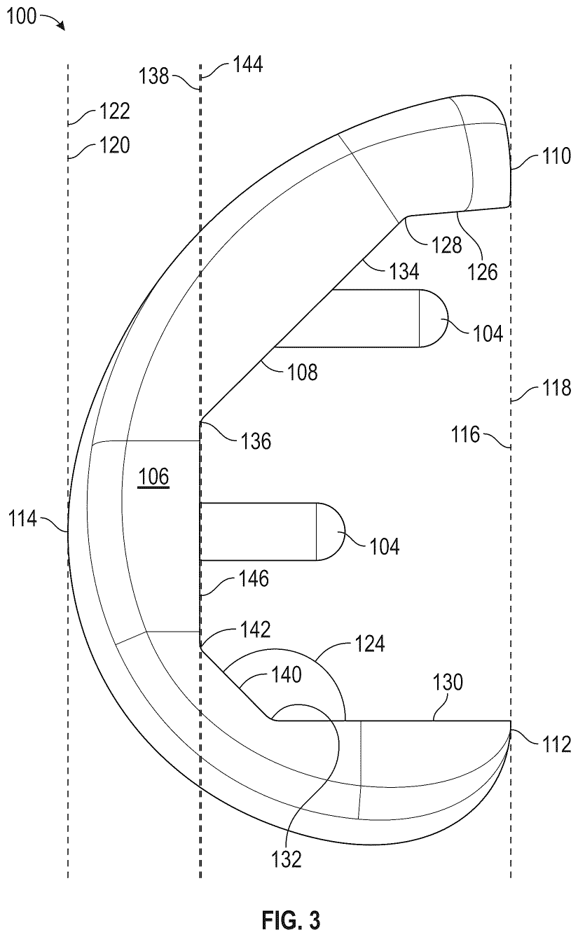

Each of FIGS. 1, 2 and 3 illustrates a first example unicompartmental femoral component arthroplasty The 100. unicompartmental arthroplasty femoral component 100 has a main body 102 and a plurality of pegs 104. The unicompartmental arthroplasty femoral component 100 illustrated is designed to be a press fit unicompartmental arthroplasty femoral component 100; however, the unicompartmental arthroplasty femoral component 100 can also be secured to a femur by an adhesive.

The main body 102 has an anterior surface 106, a posterior surface 108, a proximal end 110 joining the anterior surface 106 and the posterior surface 108, and a distal end 112 joining the anterior surface 106 and the posterior surface 108.

The anterior surface 106 has a substantially crescent shape, and an anterior intermediate point 114 that is disposed between the proximal end 110 and the distal end 112. The anterior surface 106 is a substantially smooth surface. The proximal end 110 and the distal end 112 are disposed on a first line 116 that lies on a first plane 118 that is parallel to a second plane 120 that includes a second line 122 that is tangent to the anterior intermediate point 114.

The posterior surface 108 defines a plurality of pairs of facets 124. In the illustrated embodiment, the posterior surface 108 defines five (5) pairs of facets. Although the illustrated embodiment depicts only five (5) pairs of facets, the posterior surface 108 can define any number of pairs of facets including, but not limited to, three, four, six, seven, nine, ten, or eleven. Having more pairs of facets is beneficial at least because obtuse angles allow the unicompartmental arthroplasty femoral component 100 to better form to a femur. Having at least five (5) pairs of facets is also beneficial at least because they add stability to the implant by increasing contact area. Each facet of a first pair of facets 126 of the plurality of pairs of facets 124 extends from the proximal end 110 to a first posterior intermediate point 128 that is between the proximal end 110 and the distal end 112. Each facet of a second pair of facets 130 of the plurality of pairs of facets 124 extends from the distal end 112 to a second posterior intermediate point 132 that is between the proximal end 110 and the distal end 112. Each facet of a third pair of facets 134 of the plurality of pairs of facets 124 extends from the first posterior intermediate point 128 to a third posterior intermediate point 136 on a third plane 138 that is parallel to the second plane 120. Each facet of a fourth pair of facets 140 of the plurality of pairs of facets 124 extends from the second posterior intermediate point 132 to a fourth posterior intermediate point 142 on a fourth plane 144 that is parallel to the second plane 120. Each facet of a fifth pair of facets 146 of the plurality of pairs of facets 124 extends between the third posterior intermediate point 136 and the fourth posterior intermediate point 142. Although the plurality of pairs of facets 124 are described as being parallel, the plurality of pairs of facets 124 may be slightly divergent.

The posterior surface 108 further defines a plurality of indentations 148. The plurality of indentations 148 are configured to create a porous surface 150 to allow a femur to grow into the unicompartmental arthroplasty femoral component 100. In the illustrated embodiment, the plurality of indentations 148 are cylindrical; however, the plurality of indentations 148 can be any shape including, but not limited to, rectangular, spherical, pyramidal, conical, or a plurality of undefined shapes created by a plurality of ridges. The porous surface 150 can either be formed from the posterior surface 108, or alternatively, the porous surface 150 can be porous coat that is adhered to the posterior surface 108.

The plurality of pegs 104 extend from the posterior surface 108 of the main body 102. In the illustrated embodiment, the plurality of pegs 104 comprise a first peg 152 and a second peg 154; however, any desired number of pegs can be used including, but not limited to, three, four, five, seven, eight, ten, or eleven. In the illustrated embodiment, the first peg 152 is offset from the second peg 154 relative to the first line 116. In alternative embodiments, the first peg 152 and the second peg 154 can be offset relative to a third line 156 that is perpendicular to the first line 116. In even further embodiments, the first peg 152 and the second peg 154 are not offset. It is advantageous for the first peg 152 and the second peg 154 to be offset at least because the offsetting adds stability to the unicompartmental arthroplasty femoral component 100 when they are embedded in a femur, with or without cement, and it also prevents toggling of the component.

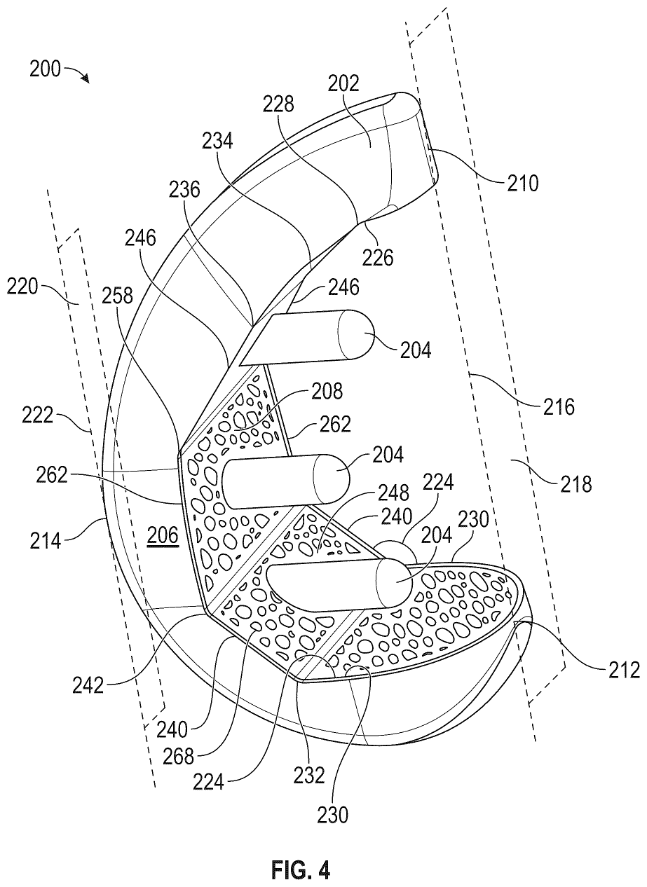

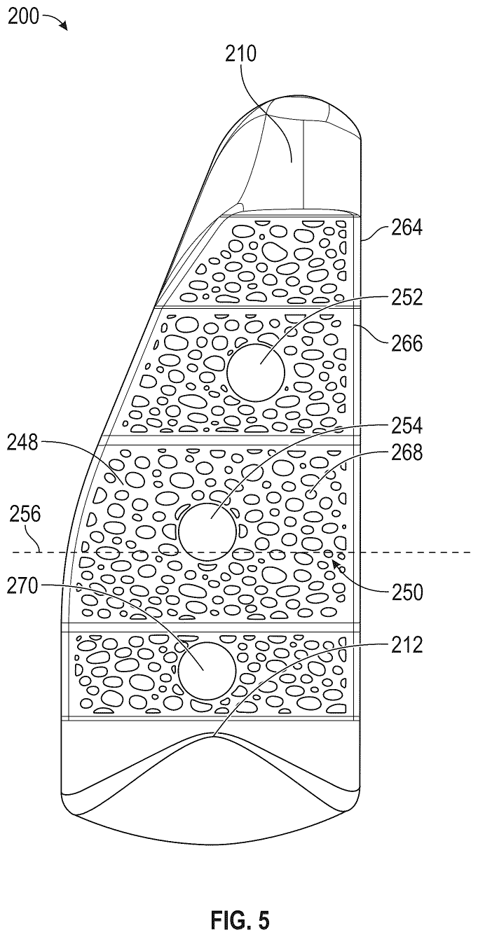

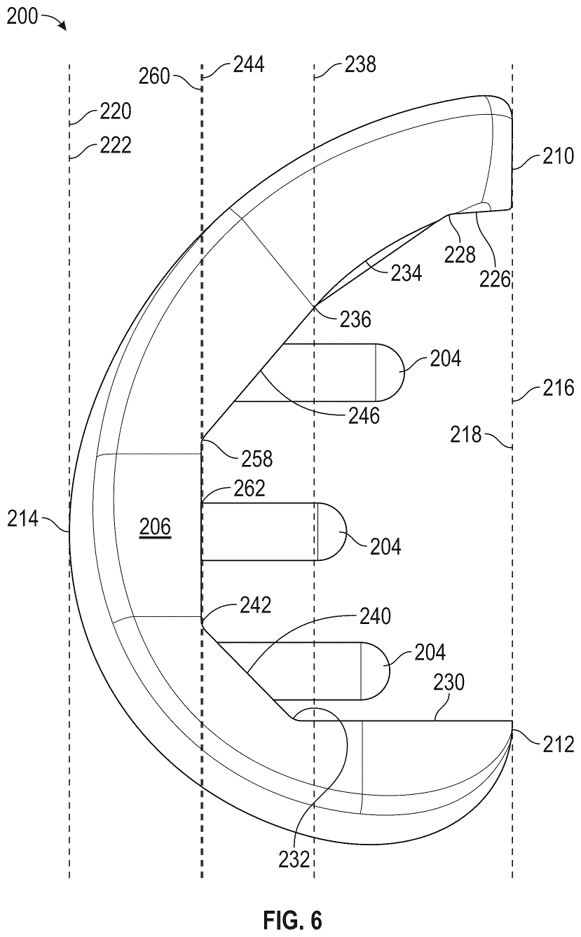

Each of FIGS. 4, 5, and 6 illustrates a second example unicompartmental arthroplasty femoral component 200. The unicompartmental arthroplasty femoral component 200 has a main body 202 and a plurality of pegs 204. The unicompartmental arthroplasty femoral component 200 illustrated is designed to be a press fit unicompartmental arthroplasty femoral component 200; however, the unicompartmental arthroplasty femoral component 200 can also be secured to a femur by an adhesive.

The main body 202 has an anterior surface 206, a posterior surface 208, a proximal end 210 joining the anterior surface 206 and the posterior surface 208, and a distal end 212 joining the anterior surface 206 and the posterior surface 208.

The anterior surface 206 has a substantially crescent shape, and an anterior intermediate point 214 that is disposed between the proximal end 210 and the distal end 212. The anterior surface 206 is a substantially smooth surface. The proximal end 210 and the distal end 212 are disposed on a first line 216 that lies on a first plane 218 that is parallel to a second plane 220 that includes a second line 222 that is tangent to the anterior intermediate point 214.

The posterior surface 208 defines a plurality of pairs of facets 224. In the illustrated embodiment, the posterior surface 208 defines six (6) pairs of facets. Although the illustrated embodiment depicts only six (6) pairs of facets, the posterior surface 208 can define any number of pairs of facets including, but not limited to, three, four, five, seven, nine, ten, or eleven. Having more pairs of facets is beneficial at least because obtuse angles allow the unicompartmental arthroplasty femoral component 200 to better form to a femur. Having at least six (6) pairs of facets is also beneficial at least because they add stability to the implant by increasing the contact area. Each facet of a first pair of facets 226 of the plurality of pairs of facets 224 extends from the proximal end 210 to a first posterior intermediate point 228 that is between the proximal end 210 and the distal end 212. Each facet of a second pair of facets 230 of the plurality of pairs of facets 224 extends from the distal end 212 to a second posterior intermediate point 232 that is between the proximal end 210 and the distal end 212. Each facet of a third pair of facets 234 of the plurality of pairs of facets 224 extends from the first posterior intermediate point 228 to a third posterior intermediate point 236 on a third plane 238 that is parallel to the second plane 220. Each facet of a fourth pair of facets 240 of the plurality of pairs of facets 224 extends from the second posterior intermediate point 232 to a fourth posterior intermediate point 242 on a fourth plane 244 that is parallel to the second plane 220. Each facet of a fifth pair of facets 246 of the plurality of pairs of facets 224 extends from the third posterior intermediate point 236 to a fifth posterior intermediate point 258 on a fifth plane 260 that is parallel to the second plane 220. Each facet of a sixth pair of facets 262 of the plurality of pairs of facets 224 extends between the fifth posterior intermediate point 258 and the fourth posterior intermediate point 242. Although the plurality of pairs of facets 224 are described as being parallel, the plurality of pairs of facets 224 may be slightly divergent.

The posterior surface 208 has a posterior surface perimeter 264 that defines a lip 266. In the illustrated embodiment, the lip 266 extends along the entirety of the posterior surface perimeter 264. However, in alternative embodiments, the lip 266 extends only a portion around the posterior surface perimeter 264. The posterior surface 208 further defines a plurality of indentations 248. The plurality of indentations 248 are configured to create a porous surface 250 to allow a femur to grow into the unicompartmental arthroplasty femoral component 200. In the illustrated embodiment, the plurality of indentations 248 includes a plurality of undefined shapes created by a plurality of ridges 268. However, the plurality of indentations 248 can be any shape including, but not limited to, cylindrical, rectangular, spherical, pyramidal, or conical. The porous surface 250 can either be formed from the posterior surface 208, or alternatively, the porous surface 250 can be porous coat that is adhered to the posterior surface 208.

The plurality of pegs 204 extend from the posterior surface 208 of the main body 202. In the illustrated embodiment, the plurality of pegs 204 comprise a first peg 252, a second peg 254, and a third peg 270; however, any desired number of pegs can be used including, but not limited to, two, four, five, seven, eight, ten, or eleven. In the illustrated embodiment, the first peg 252 is offset from the second peg 254 relative to the first line 216, the second peg 254 is offset from the third peg 270 relative to the first line 216, and the third peg 270 is offset from the first peg 252 relative to the first line 216. In alternative embodiments, the first peg 252, the second peg 254, and the third peg 270 can be offset relative to a third line 256 that is perpendicular to the first line 216. In even further embodiments, the first peg 252, the second peg 254, and the third peg 270 are not offset or only one of the plurality of pegs 204 is offset from the others relative to the first line 216. It is advantageous for the first peg 252, the second peg 254, and the third peg 270 to be offset at least because the offsetting of the pegs adds stability to the unicompartmental arthroplasty femoral component 200 when they are embedded in a femur, with or without cement, and it also prevents toggling of the component.

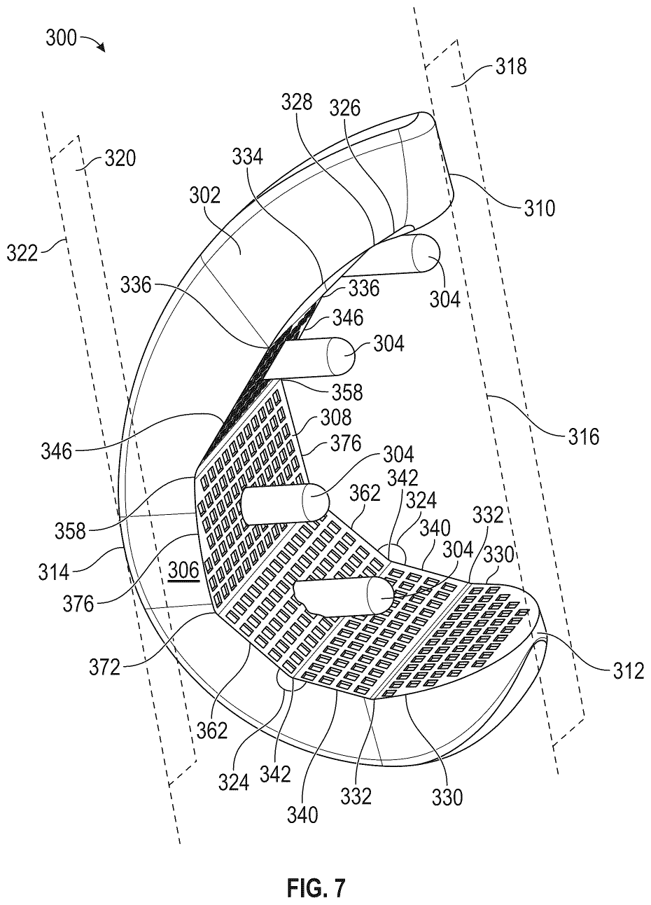

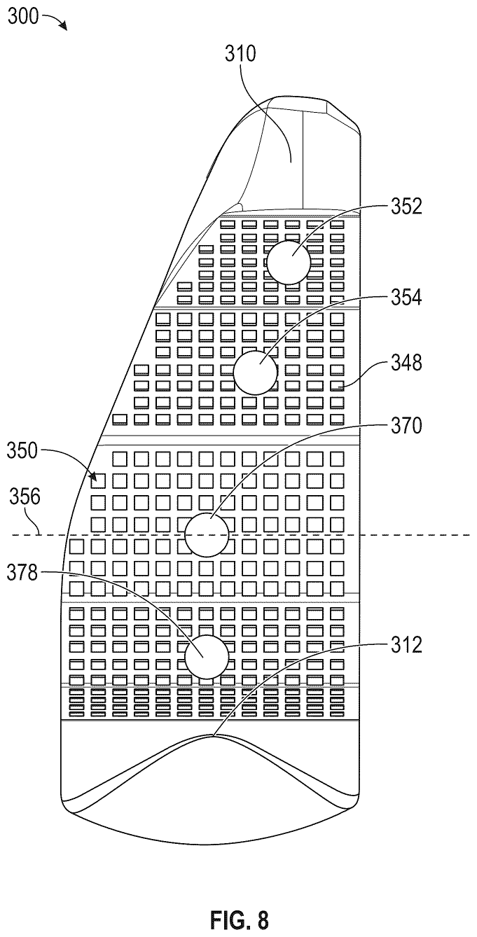

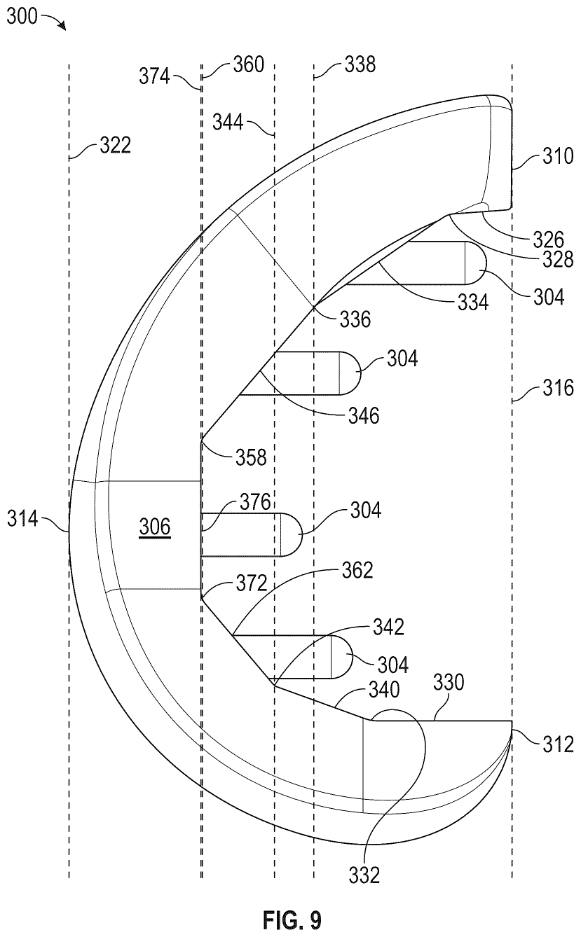

Each of FIGS. 7, 8, and 9 illustrates a third example unicompartmental arthroplasty femoral component 300. The unicompartmental arthroplasty femoral component 300 has a main body 302 and a plurality of pegs 304. The unicompartmental arthroplasty femoral component 300 illustrated is designed to be a press fit unicompartmental arthroplasty femoral component 300; however, the unicompartmental arthroplasty femoral component 300 can also be secured to a femur by an adhesive.

The main body 302 has an anterior surface 306, a posterior surface 308, a proximal end 310 joining the anterior surface 306 and the posterior surface 308, and a distal end 312 joining the anterior surface 306 and the posterior surface 308.

The anterior surface 306 has a substantially crescent shape and an anterior intermediate point 314 that is disposed between the proximal end 310 and the distal end 312. The anterior surface 306 is a substantially smooth surface. The proximal end 310 and the distal end 312 are disposed on a first line 316 that lies on a first plane 318 that is parallel to a second plane 320 that includes a second line 322 that is tangent to the anterior intermediate point 314.

The posterior surface 308 defines a plurality of pairs of facets 324. In the illustrated embodiment, the posterior surface 308 defines seven (7) pairs of facets. Although the illustrated embodiment depicts only seven (7) pairs of facets, the posterior surface 308 can define any number of pairs of facets including, but not limited to, three, four, five, six, nine, ten, or eleven. Having more pairs of facets is beneficial at least because obtuse angles allow the unicompartmental arthroplasty femoral component 300 to better form to the femur. Having at least seven (7) pairs of facets is also beneficial at least because they add stability to the implant by increasing contact area. Each facet of a first pair of facets 326 of the plurality of pairs of facets 324 extends from the proximal end 310 to a first posterior intermediate point 328 that is between the proximal end 310 and the distal end 312. Each facet of a second pair of facets 330 of the plurality of pairs of facets 324 extends from the distal end 312 to a second posterior intermediate point 332 that is between the proximal end 310 and the distal end 312. Each facet of a third pair of facets 334 of the plurality of pairs of facets 324 extends from the first posterior intermediate point 328 to a third posterior intermediate point 336 on a third plane 338 that is parallel to the second plane 320. Each facet of a fourth pair of facets 340 of the plurality of pairs of facets 324 extends from the second posterior intermediate point 332 to a fourth posterior intermediate point 342 on a fourth plane 344 that is parallel to the second plane 320. Each facet of a fifth pair of facets 346 of the plurality of pairs of facets 324 extends from the third posterior intermediate point 336 to a fifth posterior intermediate point 358 on a fifth plane 360 that is parallel to the second plane 320. Each facet of a sixth pair of facets 362 of the plurality of pairs of facets 324 extends from the fourth posterior intermediate point 342 to a sixth posterior intermediate point 372 on a sixth plane 374 that is parallel to the second plane 320. Each facet of a seventh pair of facets 376 of the plurality of pairs of facets 324 extends between the fifth posterior intermediate point 358 and the sixth posterior intermediate point 372. Although the plurality of pairs of facets 324 are described as being parallel, the plurality of pairs of facets 324 may be slightly divergent.

The posterior surface 308 further defines a plurality of indentations 348. The plurality of indentations 348 are configured to create a porous surface 350 to allow a femur to grow into the unicompartmental arthroplasty femoral component 300. In the illustrated embodiment, the plurality of indentations 348 are rectangular; however, the plurality of indentations 348 can be any shape including, but not limited to, cylindrical, spherical, pyramidal, conical, or a plurality of undefined shapes created by a plurality of ridges. The porous surface 350 can either be formed from the posterior surface 308, or alternatively, the porous surface 350 can be porous coat that is adhered to the posterior surface 308.

The plurality of pegs 304 extend from the posterior surface 308 of the main body 302. In the illustrated embodiment, the plurality of pegs 304 comprise a first peg 352, a second peg 354, a third peg 370, and a fourth peg 378; however, any desired number of pegs can be used including, but not limited to, three, four, five, six, eight, ten, or eleven. In the illustrated embodiment, the first peg 352 is offset from the second peg 354 and third peg 370 relative to the first line 316. The second peg 354 is offset from the first peg 352, the third peg 370, and the fourth peg 378 relative to the first line 316. The third peg 370 is offset from the first peg 352, the second peg 354, and the fourth peg 378 relative to the first line 316. The fourth peg 378 is offset from the second peg 354 and the third peg 370 relative to the first line 316. In alternative embodiments, the third peg 370 and the fourth peg 378 can be offset from the first peg 352 and the second peg 354 relative to the first line 316, or the second peg 354 and the fourth peg 378 can be offset from the first peg 352 and the third peg 370 relative to the first line 316. Further, the first peg 352, the second peg 354, the third peg 370, and fourth peg 378 can be offset relative to a third line 356 that is perpendicular to the first line 316. In even further embodiments, the first peg 352, the second peg 354, the third peg 370, and the fourth peg 378 are not offset. The first peg 352, the second peg 354, the third peg 370, and the fourth peg 378 can be configured in any desired arrangement and is not bound by the orientations described above. It is advantageous for at least one of the first peg 352, the second peg 354, the third peg 370 or the fourth peg 378 to be offset at least because the offsetting adds stability to the unicompartmental arthroplasty femoral component 300 when they are in embedded in a femur, with or without cement, and it also prevents toggling of the unicompartmental arthroplasty femoral component 300.

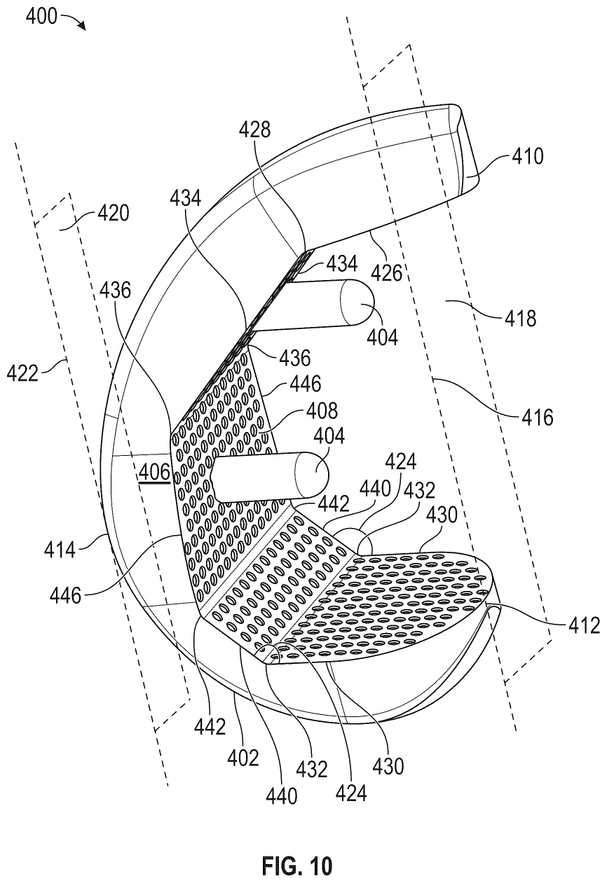

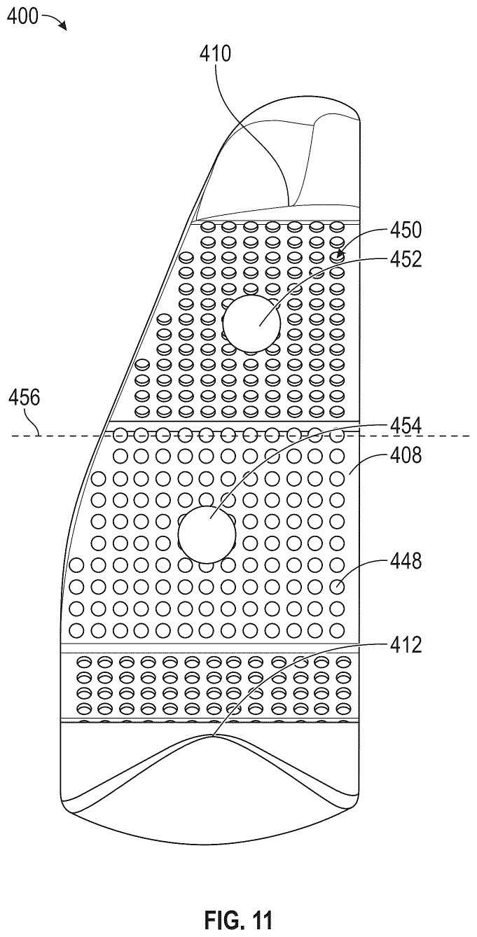

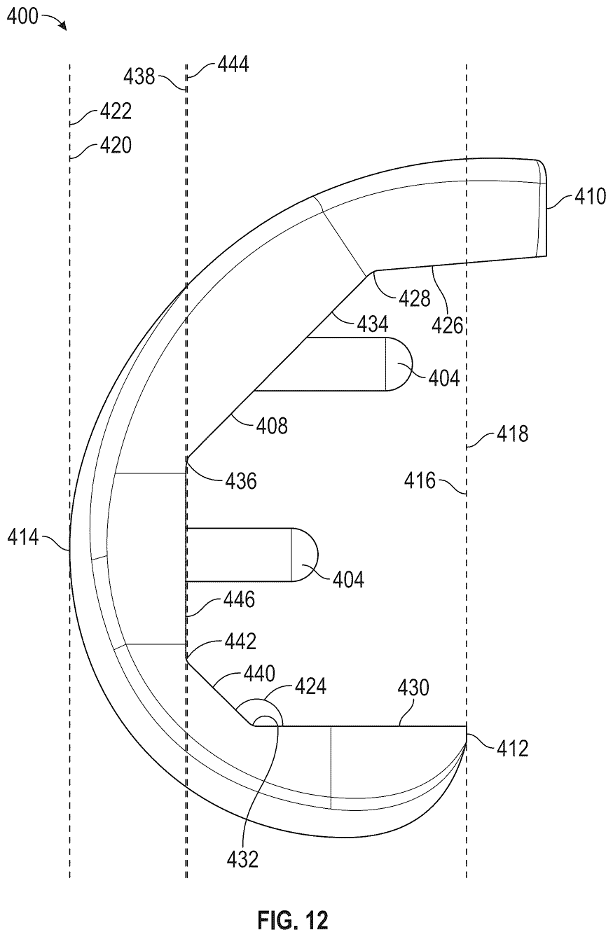

Each of FIGS. 10, 11 and 12 illustrates a fourth example unicompartmental arthroplasty femoral The component 400. unicompartmental arthroplasty femoral component 400 has a main body 402 and a plurality of pegs 404. The unicompartmental arthroplasty femoral component 400 illustrated is designed to be a press fit unicompartmental arthroplasty femoral component 400; however, the unicompartmental arthroplasty femoral component 400 can also be secured to a femur by an adhesive.

The main body 402 has an anterior surface 406, a posterior surface 408, a proximal end 410 joining the anterior surface 406 and the posterior surface 408, and a distal end 412 joining the anterior surface 406 and the posterior surface 408.

The anterior surface 406 has a substantially crescent shape, and an anterior intermediate point 414 that is disposed between the proximal end 410 and the distal end 412. The anterior surface 406 is a substantially smooth surface. The distal end 412 is disposed on a first line 416 that lies on a first plane 418 that is parallel to a second plane 420 that includes a second line 422 that is tangent to the anterior intermediate point 414. The proximal end 410 extends in a direction away from the anterior intermediate point 414 and past the first line 412 and the first plane 418.

The posterior surface 408 defines a plurality of pairs of facets 424. In the illustrated embodiment, the posterior surface 408 defines five (5) pairs of facets. Although the illustrated embodiment depicts only five (5) pairs of facets, the posterior surface 408 can define any number of pairs of facets including, but not limited to, three, four, six, seven, nine, ten, or eleven. Having more pairs of facets is beneficial at least because obtuse angles allow the unicompartmental arthroplasty femoral component 400 to better form to a femur. Having at least five (5) pairs of facets is also beneficial at least because they add stability to the implant by increasing contact area. Each facet of a first pair of facets 426 of the plurality of pairs of facets 424 extends from the proximal end 410 to a first posterior intermediate point 428 that is between the proximal end 410 and the distal end 412. Each facet of a second pair of facets 430 of the plurality of pairs of facets 424 extends from the distal end 412 to a second posterior intermediate point 432 that is between the proximal end 410 and the distal end 412. Each facet of a third pair of facets 434 of the plurality of pairs of facets 424 extends from the first posterior intermediate point 428 to a third posterior intermediate point 436 on a third plane 438 that is parallel to the second plane 420. Each facet of a fourth pair of facets 440 of the plurality of pairs of facets 424 extends from the second posterior intermediate point 432 to a fourth posterior intermediate point 442 on a fourth plane 444 that is parallel to the second plane 420. Each facet of a fifth pair of facets 446 of the plurality of pairs of facets 424 extends between the third posterior intermediate point 436 and the fourth posterior intermediate point 442. Although the plurality of pairs of facets 424 are described as being parallel, the plurality of pairs of facets 424 may be slightly divergent.

The posterior surface 408 further defines a plurality of indentations 448. The plurality of indentations 448 are configured to create a porous surface 450 to allow a femur to grow into the unicompartmental arthroplasty femoral component 400. In the illustrated embodiment, the plurality of indentations 448 are cylindrical; however, the plurality of indentations 448 can be any shape including, but not limited to, rectangular, spherical, pyramidal, conical, or a plurality of undefined shapes created by a plurality of ridges. The porous surface 450 can either be formed from the posterior surface 408, or alternatively, the porous surface 450 can be porous coat that is adhered to the posterior surface 408.

The plurality of pegs 404 extend from the posterior surface 408 of the main body 402. In the illustrated embodiment, the plurality of pegs 404 comprise a first peg 452 and a second peg 454; however, any desired number of pegs can be used including, but not limited to, three, four, five, seven, eight, ten, or eleven. In the illustrated embodiment, the first peg 452 is offset from the second peg 454 relative to the first line 416. In alternative embodiments, the first peg 452 and the second peg 454 can be offset relative to a third line 456 that is perpendicular to the first line 416. In even further embodiments, the first peg 452 and the second peg 454 are not offset. It is advantageous for the first peg 452 and the second peg 454 to be offset at least because the offsetting adds stability to the unicompartmental arthroplasty femoral component 400 when they are embedded in a femur, with or without cement, and it also prevents toggling of the component.

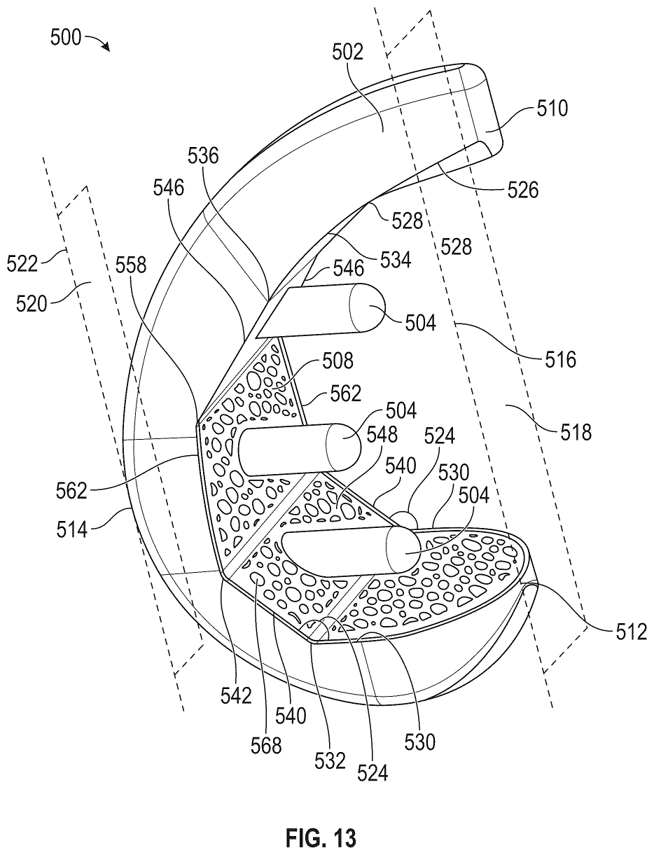

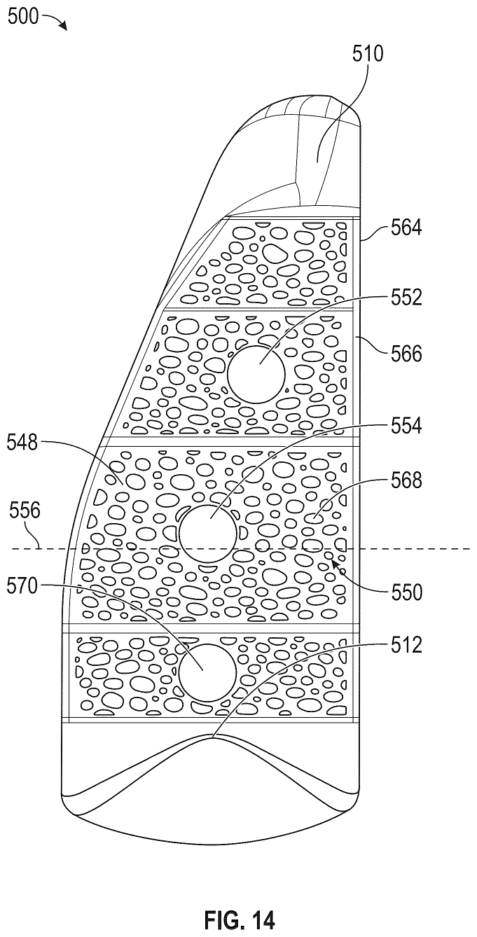

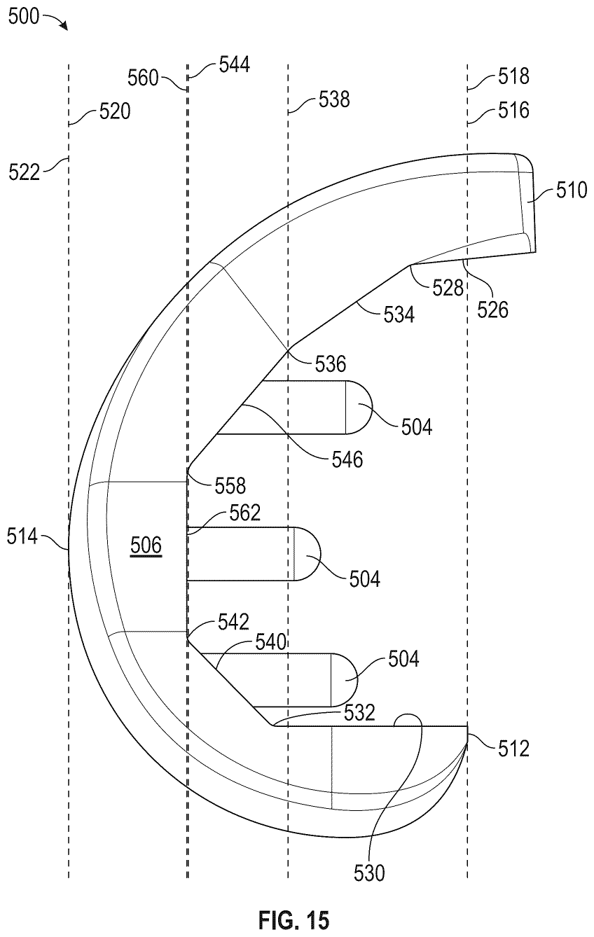

Each of FIGS. 13, 14, and 15 illustrates a second example unicompartmental arthroplasty femoral component 500. The unicompartmental arthroplasty femoral component 500 has a main body 502 and a plurality of pegs 504. The unicompartmental arthroplasty femoral component 500 illustrated is designed to be a press fit unicompartmental arthroplasty femoral component 500; however, the unicompartmental arthroplasty femoral component 500 can also be secured to a femur by an adhesive.

The main body 502 has an anterior surface 506, a posterior surface 508, a proximal end 510 joining the anterior surface 506 and the posterior surface 508, and a distal end 512 joining the anterior surface 506 and the posterior surface 508.

The anterior surface 506 has a substantially crescent shape, and an anterior intermediate point 514 that is disposed between the proximal end 510 and the distal end 512. The anterior surface 506 is a substantially smooth surface. The distal end 512 is disposed on a first line 516 that lies on a first plane 518 that is parallel to a second plane 520 that includes a second line 522 that is tangent to the anterior intermediate point 514. The proximal end 510 extends in a direction away from the anterior intermediate point 514 and past the first line 512 and the first plane 518.

The posterior surface 508 defines a plurality of pairs of facets 524. In the illustrated embodiment, the posterior surface 508 defines six (6) pairs of facets. Although the illustrated embodiment depicts only six (6) pairs of facets, the posterior surface 508 can define any number of pairs of facets including, but not limited to, three, four, five, seven, nine, ten, or eleven. Having more pairs of facets is beneficial at least because obtuse angles allow the unicompartmental arthroplasty femoral component 500 to better form to a femur. Having at least six (6) pairs of facets is also beneficial at least because they add stability to the implant by increasing the contact area. Each facet of a first pair of facets 526 of the plurality of pairs of facets 524 extends from the proximal end 510 to a first posterior intermediate point 528 that is between the proximal end 510 and the distal end 512. Each facet of a second pair of facets 530 of the plurality of pairs of facets 524 extends from the distal end 512 to a second posterior intermediate point 532 that is between the proximal end 510 and the distal end 512. Each facet of a third pair of facets 534 of the plurality of pairs of facets 524 extends from the first posterior intermediate point 528 to a third posterior intermediate point 536 on a third plane 538 that is parallel to the second plane 520. Each facet of a fourth pair of facets 540 of the plurality of pairs of facets 524 extends from the second posterior intermediate point 532 to a fourth posterior intermediate point 542 on a fourth plane 544 that is parallel to the second plane 520. Each facet of a fifth pair of facets 546 of the plurality of pairs of facets 524 extends from the third posterior intermediate point 536 to a fifth posterior intermediate point 558 on a fifth plane 560 that is parallel to the second plane 520. Each facet of a sixth pair of facets 562 of the plurality of pairs of facets 524 extends between the fifth posterior intermediate point 558 and the fourth posterior intermediate point 542. Although the plurality of pairs of facets 524 are described as being parallel, the plurality of pairs of facets 524 may be slightly divergent.

The posterior surface 508 has a posterior surface perimeter 564 that defines a lip 566. In the illustrated embodiment, the lip 566 extends along the entirety of the posterior surface perimeter 564. However, in alternative embodiments, the lip 566 extends only a portion around the posterior surface perimeter 564. The posterior surface 508 further defines a plurality of indentations 548. The plurality of indentations 548 are configured to create a porous surface 550 to allow a femur to grow into the unicompartmental arthroplasty femoral component 500. In the illustrated embodiment, the plurality of indentations 548 includes a plurality of undefined shapes created by a plurality of ridges 568. However, the plurality of indentations 548 can be any shape including, but not limited to, cylindrical, rectangular, spherical, pyramidal, or conical. The porous surface 550 can either be formed from the posterior surface 508, or alternatively, the porous surface 550 can be porous coat that is adhered to the posterior surface 508.

The plurality of pegs 504 extend from the posterior surface 508 of the main body 502. In the illustrated embodiment, the plurality of pegs 504 comprise a first peg 552, a second peg 554, and a third peg 570; however, any desired number of pegs can be used including, but not limited to, two, four, five, seven, eight, ten, or eleven. In the illustrated embodiment, the first peg 552 is offset from the second peg 554 relative to the first line 516, the second peg 554 is offset from the third peg 570 relative to the first line 516, and the third peg 570 is offset from the first peg 552 relative to the first line 516. In alternative embodiments, the first peg 552, the second peg 554, and the third peg 570 can be offset relative to a third line 556 that is perpendicular to the first line 516. In even further embodiments, the first peg 552, the second peg 554, and the third peg 570 are not offset or only one of the plurality of pegs 504 is offset from the others relative to the first line 516. It is advantageous for the first peg 552, the second peg 554, and the third peg 570 to be offset at least because the offsetting of the pegs adds stability to the unicompartmental arthroplasty femoral component 500 when they are embedded in a femur, with or without cement, and it also prevents toggling of the component.

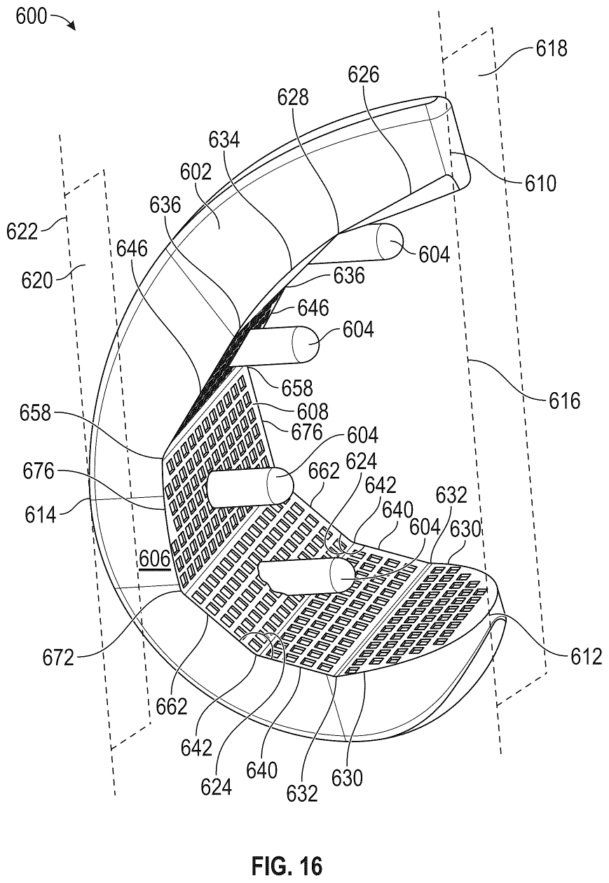

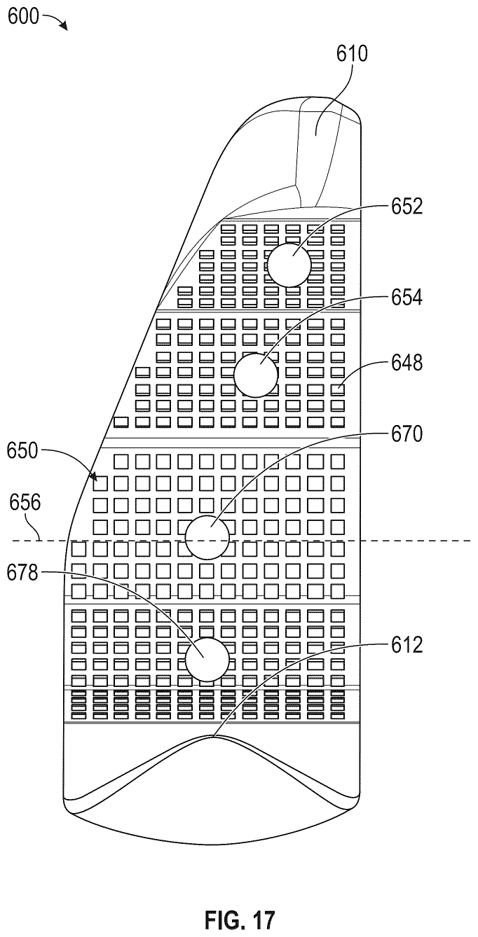

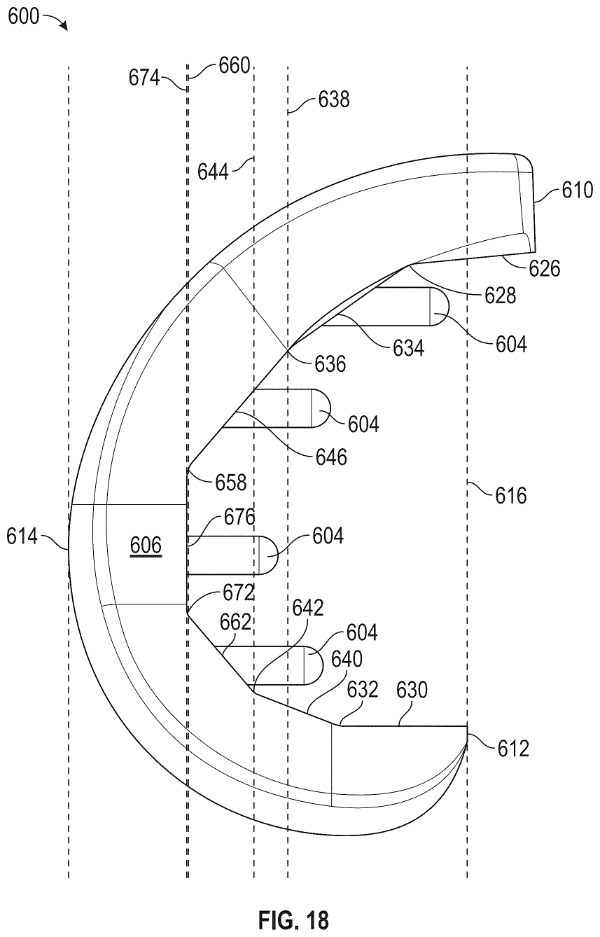

Each of FIGS. 16, 17, and 18 illustrates a sixth example unicompartmental arthroplasty femoral component 600. The unicompartmental arthroplasty femoral component 600 has a main body 602 and a plurality of pegs 604. The unicompartmental arthroplasty femoral component 600 illustrated is designed to be a press fit unicompartmental arthroplasty femoral component 600; however, the unicompartmental arthroplasty femoral component 600 can also be secured to a femur by an adhesive.

The main body 602 has an anterior surface 606, a posterior surface 608, a proximal end 610 joining the anterior surface 606 and the posterior surface 608, and a distal end 612 joining the anterior surface 606 and the posterior surface 608.

The anterior surface 606 has a substantially crescent shape and an anterior intermediate point 614 that is disposed between the proximal end 610 and the distal end 612. The anterior surface 606 is a substantially smooth surface. The distal end 612 is disposed on a first line 616 that lies on a first plane 618 that is parallel to a second plane 620 that includes a second line 622 that is tangent to the anterior intermediate point 614. The proximal end 610 extends in a direction away from the anterior intermediate point 614 and past the first line 612 and the first plane 418.

The posterior surface 608 defines a plurality of pairs of facets 624. In the illustrated embodiment, the posterior surface 608 defines seven (7) pairs of facets. Although the illustrated embodiment depicts only seven (7) pairs of facets, the posterior surface 608 can define any number of pairs of facets including, but not limited to, three, four, five, six, nine, ten, or eleven. Having more pairs of facets is beneficial at least because obtuse angles allow the unicompartmental arthroplasty femoral component 600 to better form to the femur. Having at least seven (7) pairs of facets is also beneficial at least because they add stability to the implant by increasing contact area. Each facet of a first pair of facets 626 of the plurality of pairs of facets 624 extends from the proximal end 610 to a first posterior intermediate point 628 that is between the proximal end 610 and the distal end 612. Each facet of a second pair of facets 630 of the plurality of pairs of facets 624 extends from the distal end 612 to a second posterior intermediate point 632 that is between the proximal end 610 and the distal end 612. Each facet of a third pair of facets 634 of the plurality of pairs of facets 624 extends from the first posterior intermediate point 628 to a third posterior intermediate point 636 on a third plane 638 that is parallel to the second plane 620. Each facet of a fourth pair of facets 640 of the plurality of pairs of facets 624 extends from the second posterior intermediate point 632 to a fourth posterior intermediate point 642 on a fourth plane 644 that is parallel to the second plane 620. Each facet of a fifth pair of facets 646 of the plurality of pairs of facets 624 extends from the third posterior intermediate point 636 to a fifth posterior intermediate point 658 on a fifth plane 660 that is parallel to the second plane 620. Each facet of a sixth pair of facets 662 of the plurality of pairs of facets 624 extends from the fourth posterior intermediate point 642 to a sixth posterior intermediate point 672 on a sixth plane 674 that is parallel to the second plane 620. Each facet of a seventh pair of facets 676 of the plurality of pairs of facets 624 extends between the fifth posterior intermediate point 658 and the sixth posterior intermediate point 672. Although the plurality of pairs of facets 624 are described as being parallel, the plurality of pairs of facets 624 may be slightly divergent.

The posterior surface 608 further defines a plurality of indentations 648. The plurality of indentations 648 are configured to create a porous surface 650 to allow a femur to grow into the unicompartmental arthroplasty femoral component 600. In the illustrated embodiment, the plurality of indentations 648 are rectangular; however, the plurality of indentations 648 can be any shape including, but not limited to, cylindrical, spherical, pyramidal, conical, or a plurality of undefined shapes created by a plurality of ridges. The porous surface 650 can either be formed from the posterior surface 608, or alternatively, the porous surface 650 can be porous coat that is adhered to the posterior surface 608.

The plurality of pegs 604 extend from the posterior surface 608 of the main body 602. In the illustrated embodiment, the plurality of pegs 604 comprise a first peg 652, a second peg 654, a third peg 670, and a fourth peg 678; however, any desired number of pegs can be used including, but not limited to, three, four, five, six, eight, ten, or eleven. In the illustrated embodiment, the first peg 652 is offset from the second peg 654 and third peg 670 relative to the first line 616. The second peg 654 is offset from the first peg 652, the third peg 670, and the fourth peg 678 relative to the first line 616. The third peg 670 is offset from the first peg 652, the second peg 654, and the fourth peg 678 relative to the first line 616. The fourth peg 678 is offset from the second peg 654 and the third peg 670 relative to the first line 616. In alternative embodiments, the third peg 670 and the fourth peg 678 can be offset from the first peg 652 and the second peg 654 relative to the first line 616, or the second peg 654 and the fourth peg 678 can be offset from the first peg 652 and the third peg 670 relative to the first line 616. Further, the first peg 652, the second peg 654, the third peg 670, and fourth peg 678 can be offset relative to a third line 656 that is perpendicular to the first line 616. In even further embodiments, the first peg 652, the second peg 654, the third peg 670, and the fourth peg 678 are not offset. The first peg 652, the second peg 654, the third peg 670, and the fourth peg 678 can be configured in any desired arrangement and is not bound by the orientations described above. It is advantageous for at least one of the first peg 652, the second peg 654, the third peg 670 or the fourth peg 678 to be offset at least because the offsetting adds stability to the unicompartmental arthroplasty femoral component 600 when they are in embedded in a femur, with or without cement, and it also prevents toggling of the unicompartmental arthroplasty femoral component 600.

The above-described elements of the unicompartmental arthroplasty femoral components can be made from any conventional material commonly used in implantable medical devices. Examples of the materials include, but are not limited to, titanium alloys, stainless steel, special high-strength alloys, alumina zirconia, zirconia toughed alumina, and ultra-high molecular weight polyethylene.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simply to describe and illustrate examples of the invention and are not intended to limit the scope of the invention or its protection, which is to be given the full breadth of the appended claims and any and all equivalents thereof.

We claim:

1. A unicompartmental arthroplasty femoral component, comprising:
a main body having an anterior surface, a posterior surface, a proximal end joining the anterior surface and the posterior surface, and a distal end joining the anterior surface and the posterior surface;
the anterior surface having a crescent shape and an anterior intermediate point disposed between the proximal end and the distal end, the proximal end and distal end disposed on a first line that lies on a first plane that is parallel to a second plane that includes a second line that is tangent to the anterior intermediate point;
the posterior surface defining a plurality of pairs of facets, each facet of a first pair of facets of the plurality of pairs of facets extending from the proximal end to a first posterior intermediate point that is between the proximal end and the distal end, each facet of a second pair of facets of the plurality of pairs of facets extending from the distal end to a second posterior intermediate point that is between the proximal end and the distal end, each facet of a third pair of facets of the plurality of pairs of facets extending from the first posterior intermediate point or the second posterior intermediate point to a third posterior intermediate point on a third plane that is parallel to the second plane; and
a plurality of pegs extending from the posterior surface.

2. The unicompartmental arthroplasty femoral component of claim 1, wherein the anterior surface is smooth.

3. The unicompartmental arthroplasty femoral component of claim 1, wherein at least one of the plurality of pegs is offset from at least one of the plurality of pegs relative to the first line.

4. The unicompartmental arthroplasty femoral component of claim 1, wherein at least one of the plurality of pegs is offset from at least one of the plurality of pegs relative to a third line that is perpendicular to the first line.

5. The unicompartmental arthroplasty femoral component of claim 1, further comprising a fourth pair of facets of the plurality of pairs of facets, each facet of the fourth pair of facets of the plurality of pairs of facets extending from the second posterior intermediate point to a fourth posterior intermediate point on a fourth plane that is parallel to the second plane.

6. The unicompartmental arthroplasty femoral component of claim 5, further comprising a fifth pair of facets of the plurality of pairs of facets, each facet of the fifth pair of facets of the plurality of pairs of facets extending between the third posterior intermediate point and the fourth posterior intermediate point.

7. The unicompartmental arthroplasty femoral component of claim 1, wherein the plurality of pegs comprises at least two pegs.

8. The unicompartmental arthroplasty femoral component of claim 1, wherein the plurality of pegs comprises at least three pegs.

9. The unicompartmental arthroplasty femoral component of claim 8, wherein each peg of the plurality of pegs is offset from each other relative to the first line.

10. The unicompartmental arthroplasty femoral component of claim 1, wherein the plurality of pegs comprises at least four pegs.

11. The unicompartmental arthroplasty femoral component of claim 10, wherein at one of the pegs of the plurality of pegs are offset from each other relative to the first line.

12. The unicompartmental arthroplasty femoral component of claim 10, wherein at least two of the pegs of the plurality of pegs are offset from each other relative to the first line.

13. The unicompartmental arthroplasty femoral component of claim 10, wherein at least three of the pegs of the plurality of pegs are offset from each other relative to the first line.

14. The unicompartmental arthroplasty femoral component of claim 10, wherein all of the pegs of the plurality of pegs are offset from each other relative to the first line.

15. The unicompartmental arthroplasty femoral component of claim 1, wherein the posterior surface defines a plurality of indentations.

16. The unicompartmental arthroplasty femoral component of claim 15, wherein the plurality of indentations are cylindrical.

17. The unicompartmental arthroplasty femoral component of claim 1, further comprising a porous coat adhered to the posterior surface.

18. The unicompartmental arthroplasty femoral component of claim 17, wherein the posterior surface has a posterior perimeter defining a lip.

19. A unicompartmental arthroplasty femoral component, comprising:
a main body having an anterior surface, a posterior surface, a proximal end joining the anterior surface and the posterior surface, and a distal end joining the anterior surface and the posterior surface;
the anterior surface having a crescent shape and an anterior intermediate point disposed between the proximal end and the distal end, the proximal end and distal end disposed on a first line that lies on a first plane that is parallel to a second plane that includes a second line that is tangent to the anterior intermediate point;
the posterior surface having a posterior surface perimeter, the posterior surface defining a plurality of pairs of facets and a lip, each facet of a first pair of facets of the plurality of pairs of facets extending from the proximal end to a first posterior intermediate point that is between the proximal end and the distal end, each facet of a second pair of facets of the plurality of pairs of facets extending from the distal end to a second posterior intermediate point that is between the proximal end and the distal end, each facet of a third pair of facets of the plurality of pairs of facets extending from the first posterior intermediate point or the second posterior intermediate point to a point on a third plane that is parallel to the second plane; and
a plurality of pegs extending from the posterior surface.

20. A unicompartmental arthroplasty femoral component, comprising:
a main body having an anterior surface, a posterior surface, a proximal end joining the anterior surface and the posterior surface, and a distal end joining the anterior surface and the posterior surface;
the anterior surface having a crescent shape and an anterior intermediate point disposed between the proximal end and the distal end, the proximal end and distal end disposed on a first line that lies on a first plane that is parallel to a second plane that includes a second line that is tangent to the anterior intermediate point;
the posterior surface defining a plurality of pairs of facets, each facet of a first pair of facets of the plurality of pairs of facets extending from the proximal end to a first posterior intermediate point that is between the proximal end and the distal end, each facet of a second pair of facets of the plurality of pairs of facets extending from the distal end to a second posterior intermediate point that is between the proximal end and the distal end, each facet of a third pair of facets of the plurality of pairs of facets extending from the first posterior intermediate point or the second posterior intermediate point to a point on a third plane that is parallel to the second plane; and
a plurality of pegs extending from the posterior portion, at least one of the plurality of pegs being offset from the others.
